(19) United States
(12) Patent Application Publication
(10) Pub. No.: US 2011/0054489 A1
(43) Pub. Date: Mar. 3, 2011

(54) One stroke double grip umbiilcal cord clamps

(76) Inventors: Edwin Pan, Hartsdale, NY (US); Maria Sumallo, Nanuet, NY (US); Renato Sumallo, Nanuet, NY (US)

(21) Appl. No.: 12/806,088

(22) Filed: Aug. 5, 2010

Related U.S. Application Data
(60) Provisional application No. 61/273,632, filed on Aug. 5, 2009.

(51) Int. Cl. A61B 17/125

(52) U.S. Cl. 606/120

(57) ABSTRACT

The One Stroke Double Grip Umbilical Cord Clamps comprises a specially designed three-in-one clamping tool boasting integrated cutting blades designed specifically to clamp and cut through an umbilical cord, simultaneously. Manufactured primarily of heavy duty plastic and surgical grade metal material, the One Stroke Double Grip Umbilical Cord Clamps comprises a three piece unit comprised of three, individual clamps positioned side by side and removably attached to one another. The novel feature of the One Stroke Double Grip Umbilical Cord Clamps is found in the center clamp. Lining the jaws of this clamp are two, sharpened, surgical stainless steel cutting blades designed to effortlessly slice through the umbilical cord, as it is snapped closed. As mentioned, this center clamp is removably attached to the outer clamps, thus enabling the caregiver to easily “break away” the cutting clamp, leaving the outer clamps securely attached to the umbilical cord stump and placenta cord, respectively.

### CLAIM OF PRIORITY

This patent application claims priority under 35 USC 119(e)(1) from U.S. Provisional Patent Application Ser. No. 61/273,632 filed Aug. 5, 2009, of Maria Sumallo, Renato Sumallo and Edwin Pan entitled, “One Stroke Quadruple Grip Umbilical Cord Clamps.”

### FIELD OF THE INVENTION

The present invention pertains to the field of obstetrics, and more specifically to the field of umbilical cord clamps.

### BACKGROUND OF THE INVENTION

The prior art has put forth several designs for umbilical cord clamps. Among these are:

U.S. Pat. No. 5,938,666 to Reynolds and Kurowski describes an umbilical cord clamp comprising a unitary clamp integrally structured from two U-shaped members respectively disposed from one another.

U.S. Pat. No. 6,638,282 to Ramsey et al describes an umbilical cord cutting and clamping device comprising a first and second clamp each having an upper arm and a lower arm.

U.S. Pat. No. 6,740,095 to Watson, Jr. et al describes an umbilical cord clamp and cutter for clamping and cutting an umbilical cord in one motion.

None of these prior art references describe the present invention.

### SUMMARY OF THE INVENTION

It is an object of the present invention to provide an improved umbilical cord clamp.

### BRIEF DESCRIPTION OF THE DRAWINGS

FIG. 1 is a perspective view of the umbilical cord clamp of the present invention in the closed position.

FIG. 2 is a perspective view of the umbilical cord clamp of the present invention in the open position.

FIG. 3 is a perspective view of the present invention showing the elements thereof separated from one another.

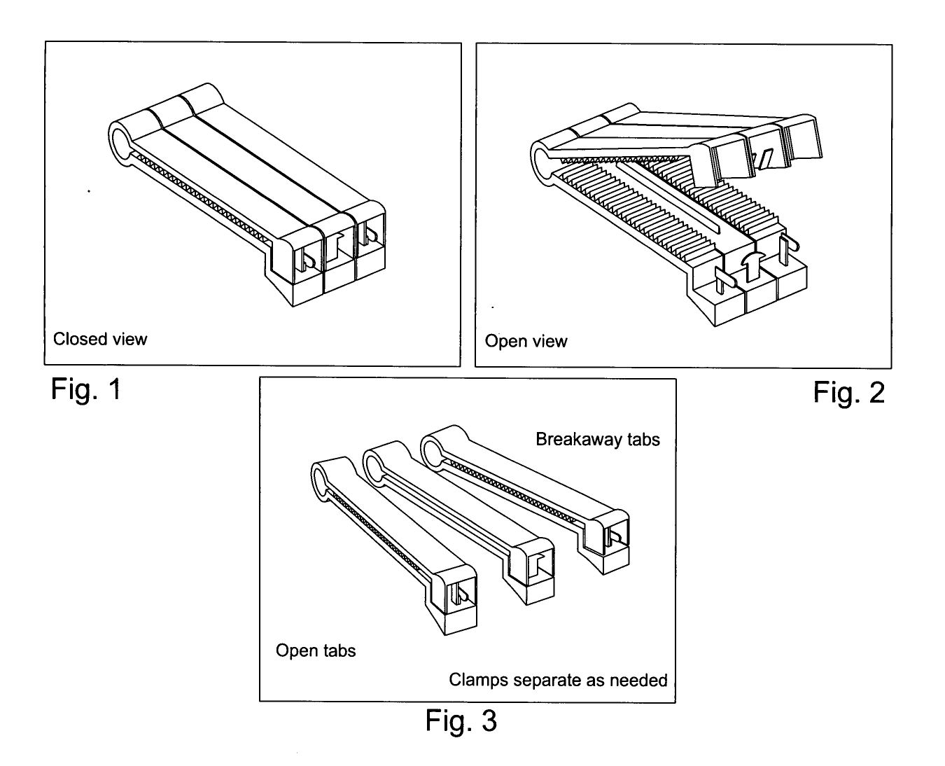

### DETAILED DESCRIPTION OF THE INVENTION

Typically, when a human baby is delivered, the umbilical cord is clamped and then carefully cut by the doctor, nurse, or in many cases, by the baby's father or a close relative. Prior to cutting the cord, a doctor will place two clamps, side by side over the cord and the cord will be cut between the two in order to minimize bleeding. Often times, such as in an emergency situation, this important procedure must be executed extremely quickly in order to enable caregivers to better administer treatment to the child or the mother.

Having recognized the potential for a more expeditious and simple means of cutting the umbilical cord following the birth of a child, the present invention, hereinafter referred to as the One Stroke Double Grip Umbilical Cord Clamps, was developed.

The One Stroke Double Grip Umbilical Cord Clamps is a specially designed three-in-one clamping tool boasting integrated cutting blades designed specifically to clamp and cut through an umbilical cord, simultaneously. Manufactured primarily of heavy duty plastic and surgical grade metal material, the One Stroke Double Grip Umbilical Cord Clamps comprises a three piece unit comprised of three, individual clamps positioned side by side and removably attached to one another.

As with traditional clamps, the One Stroke Double Grip Umbilical Cord Clamps comprises springy, triangular shaped plastic clamps with the complete set measuring approximately 2 inches in length, 1 inch in width, with the jaw span of the clamps measuring approximately 1.75 inches when opened. The two outer clamps are similar in function to traditional umbilical cord clamps and as such the jaws of the clamps are lined with rows of serrated, interlocking teeth designed to securely clamp over and grip the actual umbilical cord, with the jaws of the clamps locking closed over the cord when applied.

The novel feature of the One Stroke Double Grip Umbilical Cord Clamps however, is found in the center clamp. Lining the jaws of this clamp are two, sharpened, surgical stainless steel cutting blades designed to effortlessly slice through the umbilical cord, as it is snapped closed. As mentioned, this center clamp is removably attached to the outer clamps, thus enabling the caregiver to easily “break away” the cutting clamp, leaving the outer clamps securely attached to the umbilical cord stump and placenta cord, respectively.

The removable attachment of the three clamps comprising the present invention can be any of a variety of attachments means. These include very thin plastic which can be snapped apart, perforated plastic for easy separation or any other configuration which allows easy removal of the clamps from one another.

Another feature of the present invention is that the outer clamp can be quickly unclamped with a flip of the finger to obtain cord blood specimens and then easily re-clamped.

Importantly, the center cutting clamp comprises contrasting colors to the outer clamps, thus enabling providers to clearly discern this clamp from the other two. Further, when snapped closed, the blades of the center clamp are securely encased within the clamp itself, thus eliminating potential injury to the medical provider. To ensure the safety and well being of the infant, mother and medical personnel, the One Stroke Double Grip Umbilical Cord Clamps are manufactured in strict compliance with Occupational Health and Safety Administration (OSHA) standards for medical equipment.

Reducing the three steps necessary to cut and clamp an umbilical cord into one, quick step, the One Stroke Double Grip Umbilical Cord Clamps enables caregivers to clamp and cut through an umbilical cord simultaneously, thus greatly reducing the time necessary to complete this procedure. As such, use of this product will prove especially useful in an emergency situation when time is of the essence. Enabling providers to quickly cut and clamp an umbilical cord in order to treat newborns and mothers in distress, use of this invention potentially means the difference between a healthy child and one who suffers permanent disability or in worse case scenarios, death.

The present invention is most beneficial for use in hospitals and birthing centers, where multitudes of newborns require umbilical cord care. Additionally, the One Stroke Double Grip Umbilical Cord Clamps is well suited for use by EMTs and midwives who deliver babies in the comfort of the parent's home. Sanitary and sensible, the One Stroke Double Grip Umbilical Cord Clamps will be well received by medical professionals, midwives and new parents.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An improved umbilical cord clamp and cutting device, comprising three individual clamps positioned side by side and releasably attached to one another, wherein the central clamp comprises an integrated cutting blade to cut through an umbilical cord, at the same time and with the same motion that the two outer clamps clamp the umbilical cord.

2. The improved umbilical cord clamps of claim 1 wherein the releasable attachment comprises thin breakable plastic.

3. The improved umbilical cord clamps of claim 1 wherein the releasable attachment comprises perforated plastic.

4. The improved umbilical cord clamps of claim 1 comprising clamps having a hinged end which allows the clamps to open to about 45 degrees and a clasp end opposite the hinged end.

5. The improved umbilical cord clamps of claim 1 wherein the clasp end comprises an overlapping clasp which can be easily opened and closed by a user by an upward motion of a single finger or thumb.

6. The improved umbilical cord clamps of claim 5 wherein the device measures approximately 2 inches in length and 1 inch in width, wherein the jaw span of the clamps measures approximately 1.75 inches when opened.

7. The improved umbilical cord clamps of claim 1 wherein the two outer clamps are lined with rows of serrated, interlocking teeth designed to securely clamp over and grip the actual umbilical cord, with the jaws of the clamps locking closed over the cord when applied.

8. The improved umbilical cord clamps of claim 1 wherein the central clamp is lined with two, sharpened, surgical stainless steel cutting blades designed to effortlessly slice through the umbilical cord, as it is snapped closed.

9. The improved umbilical cord clamps of claim 1 wherein the central clamp is removably attached to the outer clamps, thus enabling the caregiver to easily “break away” the cutting clamp, leaving the outer clamps securely attached to the umbilical cord stump and placenta cord, respectively.

10. The improved umbilical cord clamps of claim 1 wherein the central clamp comprises a different color than the outer clamps, thus enabling providers to clearly discern this clamp from the other two clamps.
